(19) United States
(12) Patent Application Publication
(10) Pub. No.: US 2013/0032164 A1
(43) Pub. Date: Feb. 7, 2013

(54) DETERGENT COSMETIC COMPOSITION COMPRISING ANIONIC AND AMPHOTERIC SURFACTANTS, A HIGHLY CHARGED CATIONIC POLYMER AND A WATER-SOLUBLE SALT

(71) Applicant: Frederic SIMONET, Touquin (FR)

(72) Inventor: Frederic SIMONET, Touquin (FR)

(73) Assignee: L'OREAL, Paris (FR)

(21) Appl. No.: 13/567,673

(22) Filed: Aug. 6, 2012

Related U.S. Application Data
(63) Continuation of application No. 11/017,868, filed on Dec. 22, 2004, now abandoned.
(60) Provisional application No. 60/540,339, filed on Feb. 2, 2004.

(30) Foreign Application Priority Data
Jan. 5, 2004 (FR) 04 00027

(51) Int. Cl. A61K 8/84; A61K 8/92; A61Q 5/12; A61Q 5/02

(52) U.S. Cl. 132/202; 424/70.17

(57) ABSTRACT

The invention relates to a detergent composition containing, in a cosmetically acceptable aqueous medium, at least one anionic surfactant and at least one amphoteric surfactant chosen from (C₈-C₂₄)alkylamido(C₃-C₈)alkylbetaines, sulfobetaines, (C₈-C₂₄)alkylamido(C₆-C₈)alkylsulfobetaines, (C₈-C₂₄)alkyl amphomonoacetates, (C₈-C₂₄)alkyl amphodiacetates, (C₈-C₂₄)alkyl amphomonopropionates, (C₈-C₂₄)alkyl amphodipropionates and phosphobetaines, in an anionic surfactant(s)/amphoteric surfactant(s) weight ratio of less than or equal to 1; at least one cationic polymer with a cationic charge density of greater than 5 meq/g, and at least 1% by weight, relative to the total weight of the composition, of at least one mineral or organic water-soluble salt, the anion of this salt comprising from 1 to 7 carbon atoms, the total amount of surfactants in the composition being less than or equal to 18% by weight relative to the total weight of the composition. This composition is especially used for washing and conditioning keratin materials, for example the hair.

### REFERENCE TO PRIOR APPLICATIONS

This application is a continuation of U.S. Ser. No. 11/017,868 filed Dec. 22, 2004, and claims priority to U.S. provisional application 60/540,339 filed Feb. 2, 2004, and to French patent application 0400027 filed Jan. 5, 2004, both incorporated herein by reference.

### FIELD OF THE INVENTION

The present invention relates to a detergent cosmetic composition comprising a particular washing base, a cationic polymer of high charge density and a water-soluble salt, to the use of the said composition for washing and conditioning keratin materials, and to a cosmetic process for treating keratin materials using this composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The drawings and description are to be regarded as illustrative in nature, and not as restrictive.

### BACKGROUND OF THE INVENTION

It is common practice, for the cleansing and/or washing of keratin materials such as the hair, to use detergent compositions (such as shampoos) based essentially on standard surfactants especially of anionic, nonionic and/or amphoteric type, but more particularly of anionic type. These compositions are applied to wet hair and the lather generated by massaging or rubbing with the hands makes it possible, after rinsing with water, to remove diverse soiling initially present on the hair or the skin.

These base compositions do admittedly have good washing power, but the intrinsic cosmetic properties associated therewith however remain fairly low, especially due to the fact that the relatively aggressive nature of such a cleansing treatment can in the long term cause more or less pronounced damage to the hair fibre, associated in particular with the gradual removal of the lipids or proteins contained in or at the surface thereof.

Thus, to improve the cosmetic properties of the above detergent compositions, and more particularly of those intended to be applied to sensitized hair (i.e. hair in a damaged or embrittled state, especially due to the chemical action of atmospheric agents and/or of hair treatments such as permanent-waving, dyeing or bleaching), it is now common practice to introduce into these compositions additional cosmetic agents known as conditioners, which are intended mainly to repair or limit the harmful or undesirable effects induced by the various treatments or aggressions to which the hair fibres are more or less repeatedly subjected. These conditioners can, of course, also improve the cosmetic behaviour of natural hair.

With this aim, cationic polymers, in particular highly charged cationic polymers, may be used due to their particularly high conditioning power, especially on sensitized hair.

However, they have the drawback of forming a complex with anionic compounds and of precipitating, for example when they are introduced into a washing base comprising anionic and amphoteric surfactants.

It is thus difficult to maintain a homogeneous nature and good working qualities such as abundant lather or viscosity that is acceptable and adjustable by addition of an intermediary compound to a composition comprising such cationic polymers and a washing base.

The Applicant has found, surprisingly, that homogeneous detergent compositions comprising an appreciable amount of highly charged cationic polymers can be obtained by using at least one anionic surfactant and at least one particular amphoteric surfactant in an anionic surfactant(s)/amphoteric surfactant(s) weight ratio of less than or equal to 1, a minimum amount of at least one particular water-soluble salt and a total amount of surfactants of less than or equal to 18% by weight relative to the total weight of the composition. This composition makes it possible to overcome the drawbacks outlined above.

In addition, these compositions have acceptable viscosities (viscosity>2 Pa·s at 25° C. at a shear rate of less than or equal to 1 s⁻¹, measured using a cone-plate rheometer such as the ThermoRheo RS1 machine), which may be adjusted by simple addition of salt or of thinner according to the industrial requirements, while at the same time remaining clear.

One subject of the invention is thus a detergent cosmetic composition comprising, in a cosmetically acceptable aqueous medium, at least one anionic surfactant and at least one amphoteric surfactant as described below in an anionic surfactant(s)/amphoteric surfactant(s) weight ratio of less than or equal to 1, at least one cationic polymer with a cationic charge density of greater than 5 meq/g and at least 1% by weight of a mineral or organic water-soluble salt, the anion of this salt comprising, if it is organic, from 1 to 7 carbon atom, and comprising a total amount of surfactants of less than or equal to 18% by weight relative to the total weight of the composition.

Another subject of the invention is the use of the composition for washing and/or conditioning keratin materials such as the hair.

A subject of the invention is also a cosmetic process for treating keratin materials using the composition according to the invention.

Other subjects, characteristics, aspects and advantages of the invention will emerge even more clearly on reading the description and the various examples that follow.

According to the invention, the detergent cosmetic composition comprises, in a cosmetically acceptable aqueous medium:
at least one anionic surfactant and at least one amphoteric surfactant chosen from (C₈-C₂₄)alkylamido(C₃-C₈)alkylbetaines, sulfobetaines, (C₈-C₂₄)alkylamido(C₆-C₈)alkylsulfobetaines, (C₈-C₂₄)alkyl amphomonoacetates, (C₈-C₂₄)alkyl amphodiacetates, (C₈-C₂₄)alkyl amphomonopropionates, (C₈-C₂₄)alkyl amphodipropionates and phosphobetaines, in an anionic surfactant(s)/amphoteric surfactant(s) weight ratio of less than or equal to 1, preferably between 0.1 and 1 and better still between 0.2 and 1,
at least one cationic polymer with a cationic charge density of greater than 5 meq/g,
at least 1% by weight, relative to the total weight of the composition, of at least one mineral or organic water-soluble salt, the anion of this salt comprising, if it is organic, from 1 to 7 carbon atoms,
the total amount of surfactants in the composition being less than or equal to 18% by weight relative to the total weight of the composition.

For the purposes of the present invention, the term “detergent composition” means a composition comprising at least 4% by weight of anionic and amphoteric surfactants, and optionally of nonionic surfactants, relative to the total weight of the composition.

The term “cosmetically acceptable medium” means a medium that is compatible with any keratin material, such as the skin, the hair, the nails, the eyelashes, the eyebrows, the lips and any other area of body or facial skin, but also of pleasant odour, appearance and feel.

Advantageously, the cationic polymer is soluble in the medium.

For the purposes of the present invention, the term “water-soluble salt” means a salt with a solubility in water at 25° C. of greater than 1%, i.e. forming at this concentration a macroscopically homogeneous, transparent and isotropic medium.

Preferably, the composition is transparent.

The term “transparent composition” means a composition with a turbidity of less than or equal to 300 NTU, the NTUs being the nephelometric units for measuring turbidity.

The turbidity may be measured, for example, using a Model 2100P turbidimeter sold by the company Hach Company, the tubes used for the measurement being referenced AR397A cat 24347-06.

The calibration is performed with formazine and the measurements are performed at room temperature (20 to 25° C.). The compositions of the invention preferably have a turbidity ranging from 0.05 to 100 NTU.

As anionic surfactants that may be used in the present invention, mention may be made especially of the salts, in particular the alkali metal salts such as the sodium salts, the ammonium salts, the amine salts, the amino alcohol salts or the alkaline-earth metal salts, for example the magnesium salts, of the following types: alkyl sulfates, alkyl ether sulfates, alkylamido ether sulfates, alkylarylpolyether sulfates, monoglyceride sulfates; alkylsulfonates, alkyl phosphates alkylamidesulfonates, alkylarylsulfonates, α-olefin sulfonates, paraffin sulfonates; alkylsulfosuccinates, alkyl ether sulfosuccinates, alkylamide sulfosuccinates; alkylsulfoacetates; acylsarcosinates; and acylglutamates, the alkyl and acyl groups of all these compounds containing from 6 to 24 carbon atoms and the aryl group preferably denoting a phenyl or benzyl group.

It is also possible to use C₆-C₂₄ alkyl esters of polyglycoside carboxylic acids such as alkyl glucoside citrates, polyalkyl glycoside tartrates, and alkyl polyglycoside sulfosuccinates; alkylsulfosuccinamates, acylisethionates and N-acyltaurates, the alkyl or acyl group of all these compounds containing from 10 to 24 carbon atoms. Among the anionic surfactants that may also be used, mention may also be made of acyllactylates in which the acyl group contains from 8 to 24 carbon atoms.

In addition, mention may also be made of alkyl-D-galactosideuronic acids and salts thereof, and also polyoxyalkylenated (C₁₀-C₂₄)alkyl ether carboxylic acids, polyoxyalkylenated (C₁₀-C₂₄)alkyl(C₆-C₂₄)aryl ether carboxylic acids, polyoxyalkylenated (C₁₀-C₂₄)alkylamido ether carboxylic acids and salts thereof, in particular those containing from 2 to 50 ethylene oxide groups, and mixtures thereof.

Among the anionic surfactants mentioned above, it is preferred according to the invention to use alkyl sulfates, alkyl ether sulfates, for instance sodium lauryl ether sulfate preferably containing 2 or 3 mol of ethylene oxide, alkyl ether carboxylates, the alkyl groups generally containing from 10 to 24 carbon atoms and preferably from 10 to 16 carbon atoms, in the particular form of sodium, magnesium or ammonium salts.

The amphoteric surfactants that are suitable in the present invention are chosen from (C₁₀-C₂₄)alkylamido(C₃-C₈)alkylbetaines, sulfobetaines, (C₁₀-C₂₄)alkylamido(C₆-C₈)alkylsulfobetaines, (C₁₀-C₂₄)alkyl amphomonoacetates, (C₁₀-C₂₄)alkyl amphodiacetates, (C₁₀-C₂₄)alkyl amphomonopropionates, (C₁₀-C₂₄)alkyl amphodipropionates and phosphobetaines, and mixtures thereof.

Among the amphoteric surfactants that may be used in the composition according to the invention, mention may be made of the products sold under the name Miranol®, as described in U.S. Pat. No. 2,528,378 and U.S. Pat. No. 2,781,354 and classified in the CTFA dictionary, 3rd edition, 1982, under the names Amphocarboxyglycinate and Amphocarboxypropionate, having the respective structures (1) and (2):

Rₐ—CONHCH₂CH₂—N(R_{b})(R_{c})(CH₂COO⁻) (1)

in which:

Rₐ represents an alkyl group derived from an acid Rₐ—COOH present in hydrolysed coconut oil, a heptyl, nonyl or undecyl group,

R_{b} represents a β-hydroxyethyl group, and

R_{c} represents a carboxymethyl group;

and

R_{a′}—CONHCH₂CH₂—N(B)(E) (2)

in which:

B represents —CH₂CH₂OX′,

E represents —(CH₂)_{z}—Y′, with z=1 or 2,

X′ represents a —CH₂CH₂—COOH group or a hydrogen atom,

Y′ represents —COOH or a —CH₂—CHOH—SO₃H group,

R_{a′} represents an alkyl group of an acid R_{a′}—COOH present in coconut oil or in hydrolysed linseed oil, an alkyl group, in particular a C₁₇ alkyl group or its iso form, an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, mention may be made of the cocoamphodiacetate sold by the company Rhodia under the trade name Miranol® C2M concentrate.

The amphoteric surfactants are preferably chosen from (C₁₀-C₂₄)alkylamido(C₃-C₈)alkylbetaines and (C₁₀-C₂₄)alkyl amphodiacetates and mixtures thereof.

Preferably, the anionic surfactant(s) is (are) present in an amount ranging from 0.4% to 9% by weight and better still from 4% to 8% by weight relative to the total weight of the composition.

Preferably, the amphoteric surfactant(s) is (are) present in an amount ranging from 2% to 16% by weight and better still from 4% to 12% by weight relative to the total weight of the composition.

The composition according to the invention may also comprise surfactants other than those defined above, such as nonionic or cationic surfactants that are well known in the art.

The composition according to the invention comprises a total amount of surfactants, including the anionic, amphoteric, nonionic and cationic surfactants, of less than or equal to 18% by weight, preferably between 4% and 18% by weight and even more preferably between 4% and 15% by weight, relative to the total weight of the cosmetic composition.

The detergent cosmetic composition according to the invention comprises one or more cationic polymers whose cationic charge density is strictly greater than 5 milliequivalents per gram and preferably between 5 and 20 meq/g.

The cationic charge density of a polymer corresponds to the number of moles of cationic charges per unit of weight of polymer under conditions in which this polymer is totally ionized. It may be determined by calculation if the structure of the polymer is known, i.e. the structure of the monomers constituting the polymer and their molar or weight proportion. It may also be determined experimentally via the Kjeldahl method.

The cationic polymers with a cationic charge density of greater than 5 meq/g that may be used in accordance with the present invention may be chosen from all those already known per se as improving the cosmetic properties of the hair treated with detergent compositions, i.e. especially those described in patent application EP-A-0 337 354 and in French patent applications FR-A-2,270,846, 2,383,660, 2,598,611, 2,470,596 and 2,519,863.

Even more generally, for the purposes of the present invention, the term “cationic polymer” denotes any polymer comprising cationic groups and/or groups that may be ionized into cationic groups.

The cationic polymers are chosen from those containing units comprising primary, secondary, tertiary and/or quaternary amine groups that either may form part of the main polymer chain or may be borne by a side substituent directly attached thereto.

The cationic polymers used generally have a number-average molecular weight of between 500 and 5×10⁶ approximately and preferably between 10³ and 3×10⁶ approximately.

Among the cationic polymers that may be mentioned more particularly are polymers of the polyamine, polyamino amide and polyquaternary ammonium type. These are known products.

The polymers of the polyamine, polyamino amide and polyquaternary ammonium type that may be used in accordance with the present invention, and that may especially be mentioned, are those described in French patents Nos 2,505,348 and 2,542,997. Among these polymers, mention may be made of:

(1) quaternized or non-quaternized vinylpyrrolidone/dialkylaminoalkyl(meth)acrylate copolymers,
(2) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers are described, in particular, in French patents 2,162,025 and 2,280,361;
(3) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2,252,840 and 2,368,508;
(4) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl group contains from 1 to 4 carbon atoms and preferably denotes methyl, ethyl or propyl. Such polymers are described in particular in French patent 1,583,363.
(5) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid being between 0.8:1 and 1.4:1; the polyamino amide resulting therefrom being reacted with epichlorohydrin in a molar ratio of epichlorohydrin relative to the secondary amine group of the polyamino amide of between 0.5:1 and 1.8:1. Such polymers are described in particular in U.S. Pat. Nos. 3,227,615 and 2,961,347.
(6) cyclopolymers of alkyldiallylamine or of dialkyldiallylammonium, such as the homopolymers or copolymers containing units corresponding to formula (VI) or (VI′):

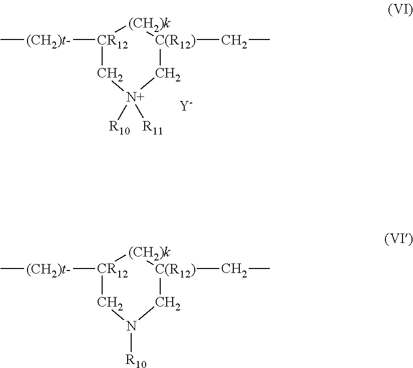

in which formulae k and t are equal to 0 or 1, the sum k+t being equal to 1; R₁₂ denotes a hydrogen atom or a methyl group; R₁₀ and R₁₁, independently of each other, denote an alkyl group containing from 1 to 22 carbon atoms, a hydroxyalkyl group in which the alkyl group preferably contains 1 to 5 carbon atoms, a lower amidoalkyl group, or R₁₀ and R₁₁ can denote, together with the nitrogen atom to which they are attached, heterocyclic groups such as piperidinyl or morpholinyl; Y⁻ is an anion such as bromide, chloride, acetate, borate, citrate, tartrate, bisulfate, bisulfite, sulfate or phosphate. These polymers are described in particular in French patent 2,080,759 and in its supplementary certificate 2,190,406.

Mention may be made, for example, of the diallyldimethylammonium chloride homopolymer sold under the name “Merquat® 100” by the company Ondeo-Nalco, and copolymers of diallyldimethylammonium chloride and of acrylamide.

(7) a diquaternary ammonium polycondensate containing repeating units corresponding to the formula:

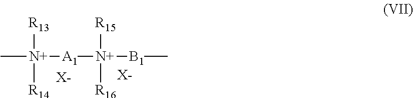

in which formula (VII):

R₁₃, R₁₄, R₁₅ and R₁₆, which may be identical or different, represent aliphatic, alicyclic or aliphatic-aryl radicals containing from 1 to 20 carbon atoms or lower aliphatic hydroxyalkyl groups, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl group substituted with a nitrile, ester, acyl or amide group or a group —CO—O—R₁₇-D or —CO—NH—R₁₇-D where R₁₇ is an alkylene and D is a quaternary ammonium group;

A₁ and B₁ represent polymethylene groups containing from 2 to 20 carbon atoms, which groups may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from an inorganic or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene group, B₁ can also denote a group —(CH₂)ₙ—CO-D—OC—(CH₂)ₙ— in which D denotes:

a) a glycol residue of formula: —O—Z—O—, where Z denotes a linear or branched hydrocarbon-based group or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, where Y denotes a linear or branched hydrocarbon-based group, or alternatively the divalent radical

—CH₂—CH₂—S—S—CH₂—CH₂—;

d) an ureylene group of formula: —NH—CO—NH—;

Preferably, X⁻ is an anion such as chloride or bromide.

These polymers generally have a number-average molecular weight of between 1,000 and 100,000.

Polymers of this type are described in particular in French patents 2,320,330; 2,270,846; 2,316,271; 2,336,434 and 2,413,907 and U.S. Pat. Nos. 2,273,780; 2,375,853; 2,388,614; 2,454,547; 3,206,462; 2,261,002; 2,271,378; 3,874,870; 4,001,432; 3,929,990; 3,966,904; 4,005,193; 4,025,617; 4,025,627; 4,025,653; 4,026,945 and 4,027,020.

It is more particularly possible to use polymers that consist of repeating units corresponding to the formula:

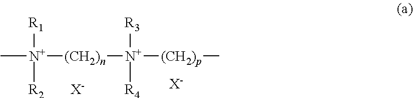

in which R₁, R₂, R₃ and R₄, which may be identical or different, denote an alkyl or hydroxyalkyl group containing from 1 to 4 carbon atoms approximately, n and p are integers ranging from 2 to 20 approximately, and X⁻ is an anion derived from an inorganic or organic acid.

One compound of formula (a) which is particularly preferred is the one for which R₁, R₂, R₃ and R₄ represent a methyl group and n=3, p=6 and X═Cl, which is known as Hexadimethrine chloride according to the INCI (CTFA) nomenclature.

(8) polyquaternary ammonium polycondensates consisting of units of formula (VIII):

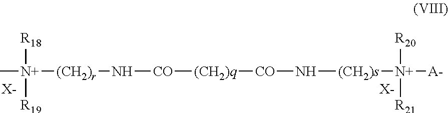

in which formula:

R₁₈, R₁₉, R₂₀ and R₂₁, which may be identical or different, represent a hydrogen atom or a methyl, ethyl, propyl, β-hydroxyethyl, β-hydroxypropyl or —CH₂CH₂(OCH₂CH₂)ₚOH group,

where p is equal to 0 or to an integer between 1 and 6, with the proviso that R₁₈, R₁₉, R₂₀ and R₂₁ do not simultaneously represent a hydrogen atom,

r and s, which may be identical or different, are integers between 1 and 6,

q is equal to 0 or to an integer between 1 and 34,

X denotes a halogen atom,

A denotes a dihalide group or preferably represents —CH₂—CH₂—O—CH₂—CH₂—.

Such compounds are described in particular in patent application EP-A-122 324.

Among these products, mention may be made, for example, of the products “Mirapol® A 15”, “Mirapol® AD1”, “Mirapol® AZ1” and “Mirapol® 175” sold by the company Miranol.

(9) homopolymers or copolymers derived from acrylic or methacrylic acids and comprising units:

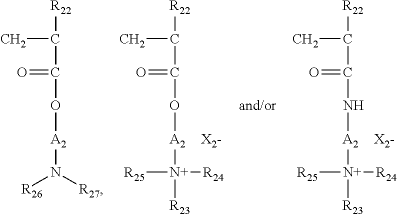

in which:

the groups R₂₂ independently denote H or CH₃, the groups A₂ independently denote a linear or branched alkyl group of 1 to 6 carbon atoms or a hydroxyalkyl group of 1 to 4 carbon atoms,

the groups R₂₃, R₂₄ and R₂₅, which may be identical or different, independently denote an alkyl group of 1 to 18 carbon atoms, or a benzyl group,

the groups R₂₆ and R₂₇ represent a hydrogen atom or an alkyl group of 1 to 6 carbon atoms,

X₂⁻ denotes an anion, for example methosulfate or halide such as chloride or bromide.

The comonomer(s) that may be used in the preparation of the corresponding copolymers belong(s) to the family of acrylamides, methacrylamides, diacetone acrylamides, acrylamides and methacrylamides substituted on the nitrogen with lower alkyls, alkyl esters, acrylic or methacrylic acids, vinylpyrrolidone or vinyl esters.

(10) Quaternary polymers of vinylpyrrolidone and of vinylimidazole.
(11) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide.

Other cationic polymers that can be used in the context of the invention are polyalkyleneimines, in particular polyethyleneimines, polymers containing vinylpyridine or vinylpyridinium units, condensates of polyamines and of epichlorohydrin, quaternary polyureylenes and chitin derivatives.

Among all the cationic polymers that may be used in the context of the present invention, it is preferred to use dialkyldiallylammonium halide homopolymers and copolymers, polyethyleneimines and polycondensates containing diquaternary ammonium or polyquaternary ammonium repeating units.

As other cationic polymers that are suitable in the present invention, mention may be made especially of cellulose-based polymers, for example the cellulose ether derivatives comprising quaternary ammonium groups described in French patent No 1,492,597.

Mention may also be made of copolymers of cellulose or of cellulose derivatives grafted with a water-soluble quaternary ammonium monomer, described especially in U.S. Pat. No. 4,131,576, such as hydroxyalkylcelluloses, for instance hydroxymethyl-, hydroxyethyl- or hydroxypropyl-celluloses grafted especially with a methacryloylethyltrimethylammonium, methacrylamidopropyltrimethylammonium or dimethyldiallylammonium salt.

Mention may also be made of guar gums.

The cationic polymers described above are preferably present in an amount ranging from 0.01% to 10% by weight, better still from 0.05% to 5% by weight, more preferably from 0.1% to 3% by weight and even more preferably from 0.5% to 4.5% by weight relative to the total weight of the composition.

The mineral or organic water-soluble salts that may be used in the present invention are chosen from water-soluble salts of monovalent or divalent metals, for example of alkali metal or alkaline-earth metals, of ammonium or of amines, and of mineral acids or of organic carboxylic acids whose anion contains from 1 to 7 carbon atoms. They preferably have a molar mass of between 25 and 650 g/mol.

Examples of such salts that may especially be mentioned include sodium chloride, potassium chloride, calcium chloride, magnesium chloride, ammonium chloride, monoethanolamine chloride, sodium citrate, ammonium citrate, magnesium sulfate and the sodium salts of phosphoric acid. The salts of monovalent metals are preferably used, and sodium chloride is particularly preferred.

The water-soluble salts are present in an amount of greater than 1% by weight, preferably in an amount ranging from 1% to 25% by weight, better still from 3% to 10% by weight and even more preferably from 4% to 8% by weight relative to the total weight of the composition.

The cosmetically acceptable aqueous medium consists of water or of a mixture of water and of at least one cosmetically acceptable solvent chosen from C₁-C₄ lower alcohols such as ethanol, isopropanol, tert-butanol or n-butanol; polyols such as glycerol, propylene glycol and polyethylene glycols; and mixtures thereof.

The pH of the compositions according to the invention is generally between 2 and 11 and preferably between 3 and 10.

The composition according to the invention may also comprise one or more standard additives that are well known in the art, such as natural or synthetic, anionic, amphoteric, zwitterionic, nonionic or cationic, associative or non-associative polymeric thickeners, non-polymeric thickeners, for instance acids or electrolytes, cationic surfactants, nacreous agents, opacifiers, dyes or pigments, fragrances, mineral, plant and/or synthetic oils, waxes, including ceramides, vitamins, UV-screening agents, free-radical scavengers, plasticizers, preserving agents or pH stabilizers.

A person skilled in the art will take care to select the optional additives and the amount thereof such that they do not harm the properties of the compositions of the present invention.

These additives are generally present in the composition according to the invention in an amount ranging from 0 to 20% by weight relative to the total weight of the composition.

The compositions in accordance with the invention may be used for washing and/or conditioning keratin materials, in particular the hair, for example as conditioning shampoos.

Another subject of the invention is a cosmetic process for treating keratin materials, such as the hair, which consists in applying an effective amount of a composition as described above to the said keratin materials, and rinsing after an optional leave-in time.

The examples that follow illustrate the present invention. The amounts indicated below are expressed as weight percentages relative to the total weight of the composition, unless otherwise mentioned.

### EXAMPLES

### Preparation of Compositions According to the Invention

Compositions 1 to 3 according to the invention were prepared using the ingredients indicated in the table below.

All the concentrations below are indicated as weight percentages of active materials.

| Composition | 1 | 2 | 3 |
|---|---|---|---|
| Sodium lauryl ether sulfate (2.2 mol of | 5% | 5% | 5% |
| ethylene oxide)⁽¹⁾ | | | |
| Disodium cocoamphodiacetate⁽²⁾ | 5% | — | — |
| Cocoylamidopropylbetaine⁽³⁾ | — | 10% | 10% |
| Poly(dimethyldiallylammonium chloride)⁽⁴⁾ | 0.5% | 0.5% | — |
| Charge density = 6.2 meq/g | | | |
| Polyethyleneimine⁽⁵⁾ | — | — | 4% |
| Charge density = 16 meq/g | | | |
| Total NaCl | 4.7% | 5.7% | 5.7% |
| Water qs | 100% | 100% | 100% |
| pH adjusted to | 7 | 7 | 7 |

⁽¹⁾sold under the trade name Texapon ® N702 by the company Cognis.
⁽²⁾sold under the trade name Miranol ® C2M by the company Rhodia Chimie.
⁽³⁾sold under the trade name Tegobetaine ® F50 by the company Goldschmidt AG.
⁽⁴⁾sold under the trade name Merquat ® 100 by the company Ondeo-Nalco.
⁽⁵⁾sold under the trade name Lupasol ® G-35 by the company BASF.

When applied to natural hair as a shampoo, these compositions give the hair a high level of disentangling, both on wet hair and on dry hair.

The above written description of the invention provides a manner and process of making and using it such that any person skilled in this art is enabled to make and use the same, this enablement being provided in particular for the subject matter of the appended claims and including a detergent cosmetic composition comprising, in a cosmetically acceptable aqueous medium:
at least one anionic surfactant and at least one amphoteric surfactant chosen from (C8-C24)alkylamido(C3-C8)alkylbetaines, sulfobetaines, (C8-C24)alkylamido(C6-C8)alkylsulfobetaines, (C8-C24)alkyl amphomonoacetates, (C8-C24)alkyl amphodiacetates, (C8-C24)alkyl amphomonopropionates, (C8-C24)alkyl amphodipropionates and phosphobetaines, in an anionic surfactant(s)/amphoteric surfactant(s) weight ratio of less than or equal to 1,
at least one cationic polymer with a cationic charge density of greater than 5 meq/g,
at least 1% by weight, relative to the total weight of the composition, of at least one mineral or organic water-soluble salt, the anion of this salt comprising from 1 to 7 carbon atoms,
the total amount of surfactants in the composition being less than or equal to 18% by weight relative to the total weight of the composition.

All references, patents, applications, tests, standards, documents, publications, brochures, texts, articles, etc. mentioned herein are incorporated herein by reference. Where a numerical limit or range is stated, all values and subranges therewithin are specifically included as if explicitly written out. As used above, the phrases “selected from the group consisting of,” “chosen from,” and the like include mixtures of the specified materials.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1: A detergent composition, comprising:
at least one anionic surfactant;
at least one amphoteric surfactant;
at least one cationic polymer with a cationic charge density of greater than 5 meq/g;
at least one mineral or organic water-soluble salt; and
a cosmetically acceptable aqueous medium;
wherein:
the at least one amphoteric surfactant is selected from the group consisting of (C8-C24)alkylamido(C3-C8)alkylbetaines, sulfobetaines, (C8-C24)alkylamido(C6-C8)alkylsulfobetaines, (C8-C24)alkyl amphomonoacetates, (C8-C24)alkyl amphodiacetates, (C8-C24)alkyl amphomonopropionates, (C8-C24)alkyl amphodipropionates and phosphobetaines;
the at least one anionic surfactant and the at least one amphoteric surfactant are present in a weight ratio of less than or equal to 1;
the at least one mineral or organic water-soluble salt is present in an amount of 4 to 8% by weight, relative to a total weight of the composition;
an anion of the at least one organic water-soluble salt comprises from 1 to 7 carbon atoms; and
a total amount of surfactants in the composition is less than or equal to 18% by weight relative to the total weight of the composition.

2: The detergent composition according to claim 1, wherein the at least one anionic surfactant and the at least one amphoteric surfactant are present in a weight ratio of between 0.1 and 1.

3: The detergent composition according to claim 1, wherein the total amount of surfactants in the composition is between 4% and 18% by weight relative to the total weight of the composition.

4: The detergent composition according to claim 1, wherein the anionic surfactant is selected from the group consisting of:
alkali metal salts, ammonium salts, amine salts, amino alcohol salts and alkaline-earth metal salts, of the following compounds: alkyl sulfates, alkyl ether sulfates, alkylamido ether sulfates, alkylarylpolyether sulfates, monoglyceride sulfates, alkylsulfonates, alkyl phosphates, alkylamidesulfonates, alkylarylsulfonates, α-olefin sulfonates, paraffin sulfonates, alkylsulfosuccinates, alkyl ether sulfosuccinates, alkylamide sulfosuccinates, alkylsulfoacetates, acylsarcosinates and acylglutamates, the alkyl and acyl groups of the compounds containing from 6 to 24 carbon atoms and the aryl group preferably denoting a phenyl or benzyl group;
C6-C24 alkyl esters of polyglycoside carboxylic acids; alkylsulfosuccinamates, acylisethionates and N-acyltaurates, the alkyl or acyl group of all these compounds containing from 12 to 20 carbon atoms; and
alkyl-D-galactosideuronic acids, polyoxyalkylenated (C₁₀-C₂₄)alkyl ether carboxylic acids, polyoxyalkylenated (C₁₀-C₂₄)alkyl(C₆-C₂₄)aryl ether carboxylic acids, polyoxyalkylenated (C₁₀-C₂₄)alkylamido ether carboxylic acids, and salts thereof.

5: The detergent composition according to claim 1, wherein the anionic surfactant comprises at least one sodium, magnesium or ammonium salt of at least one member selected from the group consisting of alkyl sulfates, alkyl ether sulfates, and alkyl ether carboxylates, the alkyl groups containing from 10 to 24 carbon atoms.

6: The detergent composition according to claim 1, wherein the amphoteric surfactant is selected from the group consisting of (C10-C24)alkylamido(C3-C8)alkylbetaines and (C10-C24)alkyl amphodiacetates.

7: The detergent composition according to claim 1, wherein the cationic polymer with a charge of greater than 5 meq/g is soluble in the composition.

8: The detergent composition according to claim 7, wherein the composition is transparent.

9: The detergent composition according to claim 1, wherein the cationic polymer has a charge density of between 5 and 20 meq/g.

10: The detergent composition according to claim 1, wherein the cationic polymer is selected from the group consisting of dialkyldiallylammonium halide homopolymers and copolymers, polyethyleneimines and polycondensates containing diquaternary ammonium or polyquaternary ammonium repeating units.

11: The detergent composition according to claim 1, wherein the composition comprises the cationic polymer in an amount of between 0.01% and 10% by weight relative to the total weight of the composition.

12: The detergent composition according to claim 1, wherein the water-soluble salt has a molar mass of between 25 and 650 g/mol.

13: The detergent composition according to claim 1, wherein the water-soluble salt is selected from the group consisting of sodium chloride, potassium chloride, calcium chloride, magnesium chloride, ammonium chloride, monoethanolamine chloride, sodium citrate, ammonium citrate, magnesium sulfate and the sodium salts of phosphoric acid.

14: The detergent composition according to claim 1, wherein the cosmetically acceptable aqueous medium consists of water or of a mixture of water and at least one organic solvent.

15: The detergent composition according to claim 1, wherein the composition further comprises at least one additive selected from the group consisting of natural or synthetic, anionic, amphoteric, zwitterionic, nonionic or cationic, associative or non-associative polymeric thickeners, non-polymeric thickeners, cationic surfactants, nacreous agents, opacifiers, dyes or pigments, fragrances, mineral, plant and/or synthetic oils, waxes, vitamins, UV-screening agents, free-radical scavengers, plasticizers, preserving agents and pH stabilizers.

16: A method, comprising applying the composition of claim 1 to keratin materials.

17: The method of claim 16, for washing and/or conditioning keratin materials.

18: A process for treating keratin material, comprising the application of an effective amount of a composition according to claim 1 to said material, and rinsing after an optional leave-in time.

19: The detergent composition according to claim 1, wherein the at least one anionic surfactant and the at least one amphoteric surfactant are present in a weight ratio of between 0.2 and 1.
